(19) United States
(12) Patent Application Publication
(10) Pub. No.: US 2026/0024618 A1
(43) Pub. Date: Jan. 22, 2026

(54) GROWTH SIGNALING AUTONOMY IN CIRCULATING TUMOR CELLS AIDING METASTATIC SEEDING

(71) Applicant: The Regents of the University of California, Oakland, CA (US)

(72) Inventors: Saptarshi Sinha, San Diego, CA (US); Pradipta Ghosh, San Diego, CA (US)

(21) Appl. No.: 19/117,892

(22) PCT Filed: Oct. 5, 2023
(86) PCT No.: PCT/US2023/034538

§ 371 Date: Apr. 2, 2025

Related U.S. Application Data
(60) Provisional application No. 63/378,625, filed on Oct. 6, 2022.

(51) Int. Cl. G16B 25/10; C12Q 1/6886; G16B 40/30; G16H 20/10; G16H 50/30

(52) U.S. Cl. CPC G16B 25/10; C12Q 1/6886; G16B 40/30; G16H 20/10; G16H 50/30

(57) ABSTRACT

Methods for analyzing the metastatic potential of circulating tumor cells (CTCs). Methods including analyzing growth signaling autonomy, methods for determining the likelihood of tumor metastases, and methods of treating a cancer determined to be likely to metastasize.

### CROSS-REFERENCE TO RELATED APPLICATIONS

This application claims priority to U.S. Provisional Application No. 63/378,625 filed on Oct. 6, 2022, the entire contents of which are incorporated by reference.

### GOVERNMENT SPONSORSHIP

This invention was made with Government support under Grant Nos. R01CA238042, R01CA100768, and R01CA160911, awarded by the National Institutes of Health (NIH). The Government has certain rights in the invention.

### TECHNICAL FIELD

The present invention relates to growth signaling autonomy in circulating tumor cells for initiating breast cancer metastases.

### BACKGROUND

Metastatic breast cancer (MBC), which involves colonization of distant sites, remains a fatal disease. While approximately 5-10% of patients are diagnosed with MBC at initial diagnosis, about 20-30% of stage I-III patients will eventually progress to MBC. Hence, understanding which cells ‘seed’ metastases in MBC is of paramount importance to improve the management and outcome of these patients.

Metastasis begins with the intravasation of cancer cells from primary tumors, either as single or in clusters, into the systemic circulation. These circulating tumor cells (CTCs) must then extravasate from the blood stream and disseminate to distant tissues, where they either remain dormant or give rise to metastases¹⁻³. However, for CTCs to ‘seed’ metastases, they must survive despite the loss of anchorage to the matrix, exposure to the immune system and hemodynamic shear forces. The past two decades has witnessed significant technological leaps to help detect, enumerate and characterize CTCs^{4,5}; their ability to initiate metastases has been correlated with their abundance⁶ as well as key phenotypic properties, e.g., enhanced protein translation⁷, proliferation^{7,8}, epithelial-mesenchymal plasticity⁹⁻¹¹, epithelial-type CTCs (with restricted mesenchymal transition^{7,12}), CTC ½ life¹³, CTCs that display cell-cell adhesion and circulate as clusters¹⁴⁻¹⁷, the presence of platelets¹⁶ and immune cells (such as neutrophils⁸) in those clusters, hypoxia¹⁸ and circadian rhythm (nighttime worse than day¹⁹). Studies using MBC-patient derived CTCs in xenotransplantation models have also revealed that the process by which CTCs initiate metastases is highly inefficient (2.7% efficiency²⁰), implying that only a fraction of CTCs are endowed with tumorigenic and metastatic functionality. However, further research is required to determine the objective molecular measurements of the metastatic potential of CTCs.

### SUMMARY OF THE INVENTION

Disclosed herein are methods for analyzing the metastatic potential of circulating tumor cells (CTCs), particularly in the context of breast cancer.

In embodiments, the disclosure provides a method for analyzing growth signaling autonomy, where the method includes defining at least one key feature of an autonomous cell line in a biological sample from a subject, and assessing the role of the at least one key feature of the autonomous cell line during tumor progression. In some embodiments, the at least one key feature is a transcriptome, a proteome, or a phenome. In some embodiments, growth signaling autonomy includes a presence of circulating tumor cells (CTCs). In some embodiments, the presence of CTCs indicates an increased likelihood of metastases.

In embodiments, a research tool for predicting tumor metastases is provided, where the tool utilizes a method for analyzing growth signaling autonomy. In some embodiments, the method includes defining at least one key feature of an autonomous cell line, and assessing the role of the at least one key feature of the autonomous cell line during tumor progression.

In embodiments, a method for determining the likelihood of tumor metastases is provided, where the method includes performing RNA sequencing on a biological sample from a subject to identify differentially expressed genes in autonomous cells, determining an expression composite score for each of the differentially expressed genes, and using the composite score to assess the autonomy of the cells, where autonomous cells are more likely to metastasize. In some embodiments, autonomy includes a presence of circulating tumor cells (CTCs). In some embodiments, the presence of CTCs indicates an increased likelihood of metastases.

In embodiments, the disclosure provides that the methods further comprise methods of treatment of a cancer comprising determining the likelihood of tumor metastases as described herein, and administering to a subject determined to have an increased likelihood of tumor metastasis an effective amount of a treatment for increased metastasis of the cancer as known in the art. The effective amount to be administered is determined based on the type of cancer and the degree of determined increase in likelihood of tumor metastasis as described herein.

### BRIEF DESCRIPTION OF THE DRAWINGS

FIG. 1. Approaches, key findings, and conclusions.

FIGS. 2A-2G. Growth signaling autonomy is required for the induction or maintenance of stemness. FIG. 2A. Schematic displays study design. Autocrine autonomy-endowed cells are compared against cells in which such autonomy is disabled (autonomy-inept) by depletion of GIV (GIV-KO). Cells are grown in the presence or absence of exogenous growth factors (0% FBS) to study the biological and translational relevance of autocrine autonomy in breast cancers. FIGS. 2B-2F. Violin plots display the single (FIG. 2B) or composite (FIGS. 2C-2F) score of selected gene signatures. P values based on Welch's T-test, comparing 10% vs 0% growth conditions in WT (blue greyscales) and KO (red greyscales) cells. Blue and red greyscales font for p values indicate, significant up-or down-regulation, respectively. FIG. G. Expression of various stemness-associated gene signatures and clinically used breast cancer gene signatures, in parental (WT) vs GIV-KO (KO, by CRISPR) VIDA MB231 cells grown in 10% or 0% FBS is visualized as bubble plots of ROC-AUC values (radius of circles are based on the ROC-AUC) demonstrating the direction of gene regulation (Up, red greyscales; Down, blue greyscales) for the classification of WT and KO samples in 10% and 0% FBS conditions based on the indicated gene signatures (bottom). BCI, breast cancer index. Nos. indicate PMIDs.

FIGS. 3A-3J. GIV-dependent growth signaling autonomy is required for the induction of cancer cell stemness. Violin plots display the composite score of selected genes and/or signatures showcased as bubble plots. P values based on Welch's T-test, comparing 10% vs 0% growth conditions in WT (blue greyscales) and KO (red greyscales) cells. Blue and red greyscales font for p values indicate significant up-or downregulation, respectively.

FIGS. 4A-4E. Growth signaling autonomy is required for the induction of molecular programs for EMT, re-epithelization and epithelial-mesenchymal plasticity (EMP). FIG. 4A. Violin plots display the composite score of various gene signatures in parental (WT) and GIV-depleted (GIV-KO) MDA MB-231 cells, which are induced during epithelial cell transition into partial (EM1, EM2, EM3) or full (M1 and M2) EMT states. FIG. 4B. Violin plots display the composite score of various gene signatures in parental (WT) and GIV-depleted (GIV-KO) MDA MB-231 cells, which are induced during the re-epithelization of partial (EM1, EM2, EM3) or full (M1 and M2) EMT states. FIGS. 4C-4E. Schematic (FIG. 4C) summarizes the study design that resulted in the discovery of the pan-cancer transcriptional census of epithelial mesenchymal plasticity (EMP) derived by single cell sequence³³. Violin plots (FIGS. 4D-4E) display the composite score of the EMP signature in parental (WT) and GIV-depleted (GIV-KO) MDA MB-231 cells. FIG. D shows the expression profile of all 328 genes in the EMP signature, representing various cells in the tumor microenvironment. FIG. E shows the expression profile of a subset of 128 genes in the EMP signature that is specifically enriched in cancer cells.

FIGS. 5A-5F. GIV-dependent growth signaling autonomy is required for both EMT and MET programs that are essential during distinct steps of tumor metastasis. FIGS. 5A-5E. Violin plots display the composite score of various gene signatures in parental (WT) and GIV-depleted (GIV-KO) MDA MB-231 cells, which capture the continuum of epithelial and mesenchymal states that aid in the metastatic spread of breast cancers. From left to right: EMT in primary tumors (FIGS. 5A-5B), to EMT and MET transition capability endowed oligoclonal tumor cell clusters in circulation (CTCs) that have ˜23-50-fold increased metastatic potential¹³ (FIG. 5C), to the MET-predominant process (FIG. 5D) of CTC re-epithelialization associated junctional signatures (FIG. 5E). p values based on Welch's T-test, comparing 10% vs 0% growth conditions in WT (blue) and KO (red) cells. Blue and red font for p values indicate, significant up- or downregulation, respectively. FIG. 5F. Summary and working model: Schematic displays the epithelial tomesenchymal (EMT) and mesenchymal to epithelial (MET) transition states in primary (left) or metastatic (right) tumors, and the transitioning state in CTC clusters.

FIGS. 6A-6F. GIV-dependent autonomy is required for growth in growth factor-restricted conditions, resilience to chemotherapeutics and metastatic seeding. FIGS. 6A-6C. Images (FIG. 6A) display representative fields acquired by light microscopy from soft-agar colony growth assays on luciferase-expressing parental (WT) and GIV-KO MDA-MB-231 cells, conducted in serum-restricted conditions. Scale bar=30 μm. Bar plots (FIG. 6B) display the number of colonies/high-power field (HPF) of parental (WT) and GIV-KO MDA-MB-231 cells, quantified using images in 1% and 0.2% FBS. Bar graphs (FIG. 6C) display the average photon flux emitted from colonies in above explained experimental setup. Results (FIGS. 6B-6C) are representative of 3 biological repeats, and 20-40 HPFs were quantified in each repeat. Error bars (FIG. 6C) represent S.E.M. p values were calculated by two-tailed t-test. FIG. 6D. IC50 values for three commonly used chemotherapeutic drugs, displayed as a heatmap. FIGS. 6E-6F. Representative whole mouse bioluminescence images (FIG. 6E) of mice acquired on day #22 after intracardiac injection of serum-deprived parental (WT) and GIV-KO MDA-MB-231 cells (FIG. 6A). The logarithmic pseudo-color scale depicts the range of bioluminescence values with red greyscales being the highest and blue greyscales the lowest. Bar graphs (FIG. 6F) display mean values±S.E.M. for area-under-the-curve (AUC) for total bioluminescence from days 1-29 after intracardiac injection for each group. N=5 mice per condition.

FIGS. 7A-7D. GIV is sufficient for therapeutic resistance (to anti-estrogen therapy) and growth factor-restricted proliferation. FIG. 7A. Line graph displays % survival of parental MCF7 cells and clones of the same stably expressing GIV (introduced by Tol2 transposon) after challenge with the indicated concentrations of the selective estrogen receptor blocker (Fulvestrant), as determined by MTT assays. Error bars represent S.E.M (n=4 biological repeats; each with 3 technical repeats). p values were calculated by one way ANOVA at a given concentration of drug. Only significant values are displayed using standard code (*p<=0.05, **p<=0.01, ***p<=0.001). FIG. 7B. Bar graphs display the proportion of surviving cells when challenged with 4 commonly used drugs at 102 nM dose. Error bars represent S.E.M. P values were calculated by one-way ANOVA at a given concentration of drug. Only significant values are displayed. FIGS. 7C-7D. Images (FIG. 7C) display representative fields acquired by light microscopy from soft-agar colony growth assays on luciferase expressing parental (MCF7) and GIV-expressing (MCF7-GIV) MCF7 cells, conducted in serumrestricted conditions. Scale bar=30 μm. Bar graph compares the viability ratio of the cells in 0.2% vs 1% serum quantified using the average photon flux emitted from the colonies (FIG. 7C). Error bars represent S.E.M (n=3 biological repeats; each with 3 technical repeats). p values were calculated by one-tailed t-test.

FIGS. 8A-8I. Genes and proteins differentially expressed between autonomy-endowed (WT) and-inept (GIV-KO) MDA-MB-231 cells. FIG. 8A. Heatmap displays DEGs (29 up- and 3 down-regulated; LogFC>5, pAdj<0.01) in MDA MB-231 parental (WT) cells compared to its GIV-depleted (KO by CRISPR) counterparts grown in 0% FBS for 16 h. FIG. 8B.

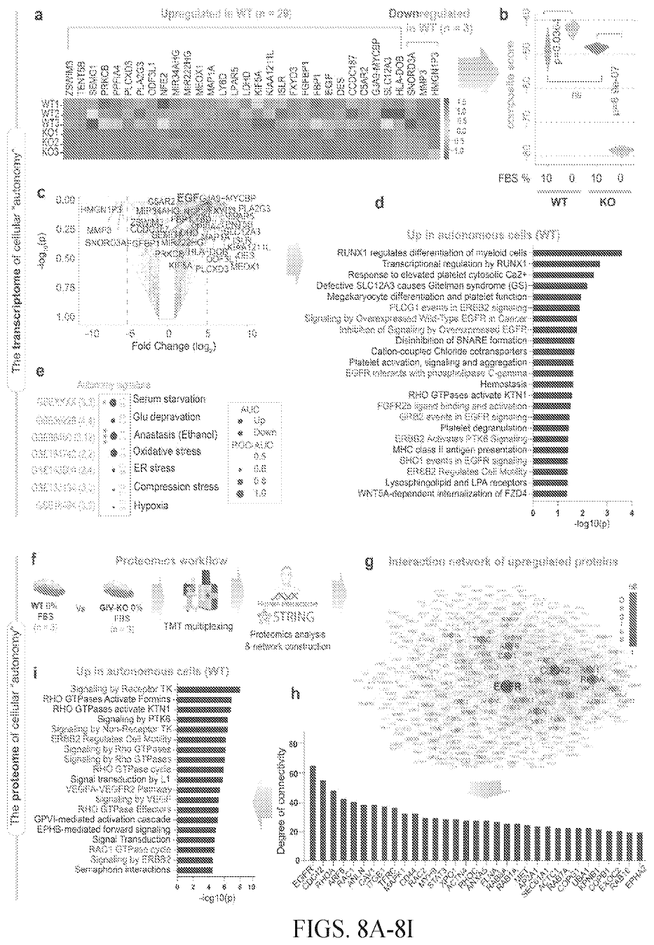

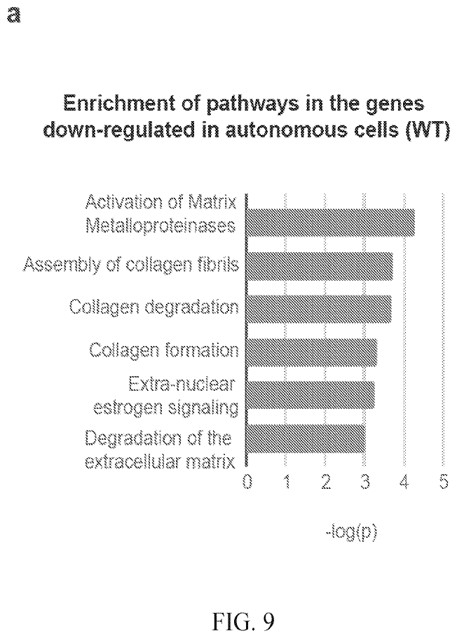

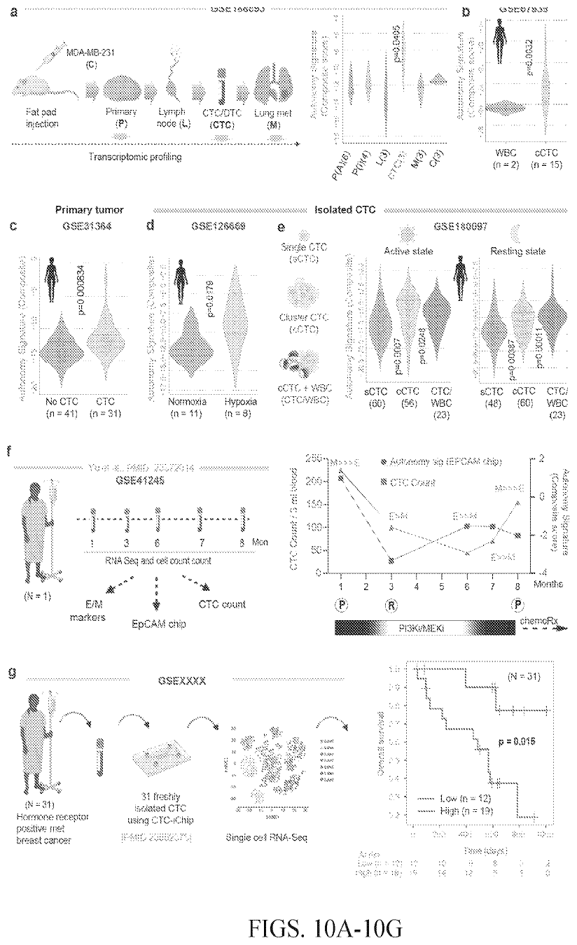

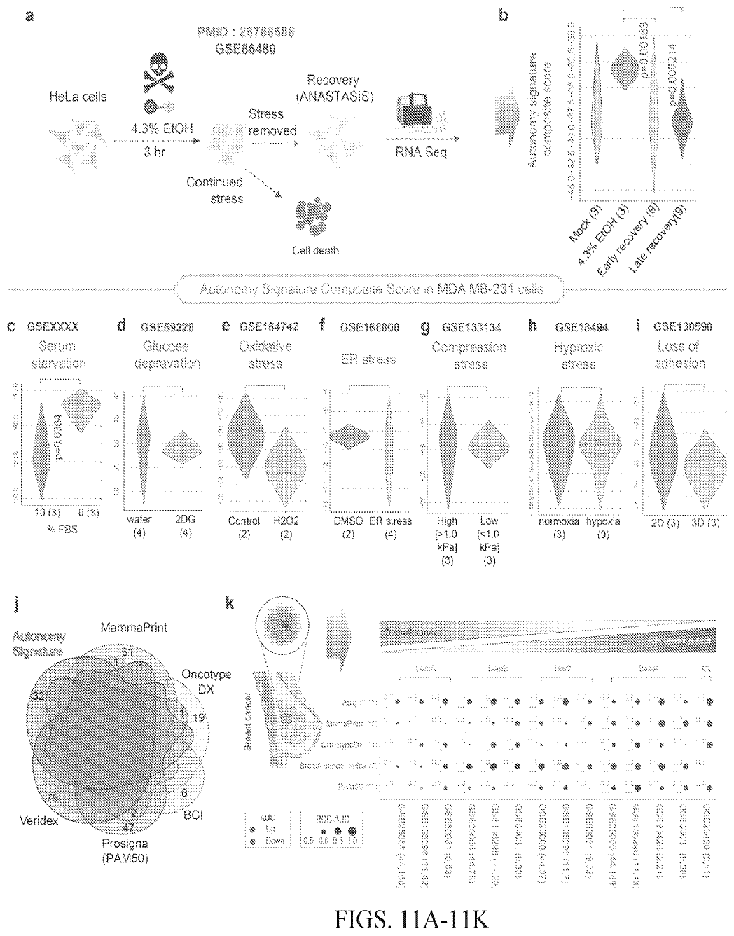

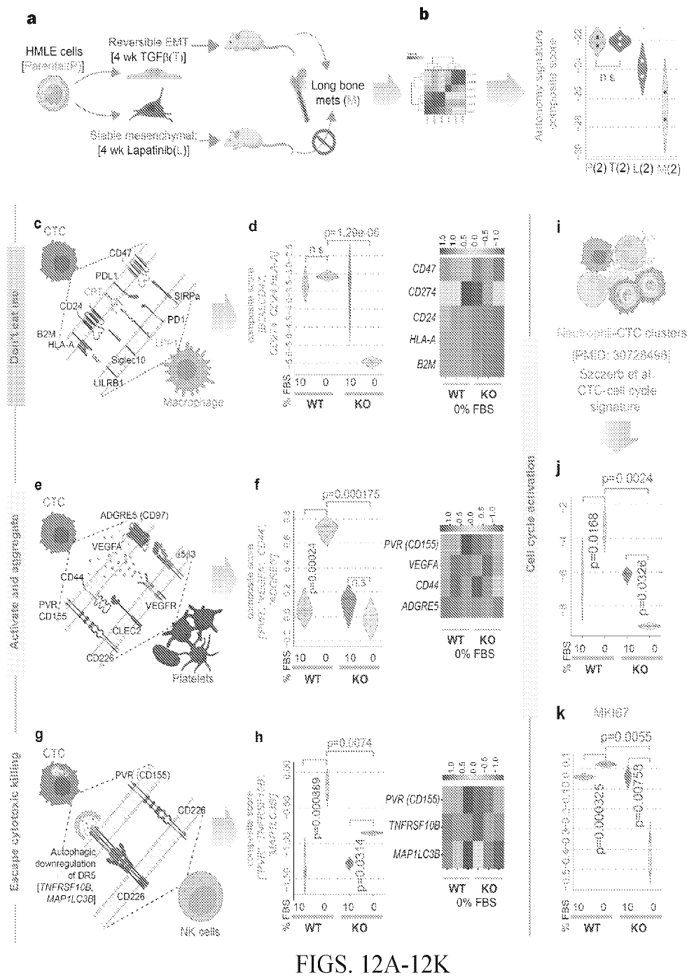

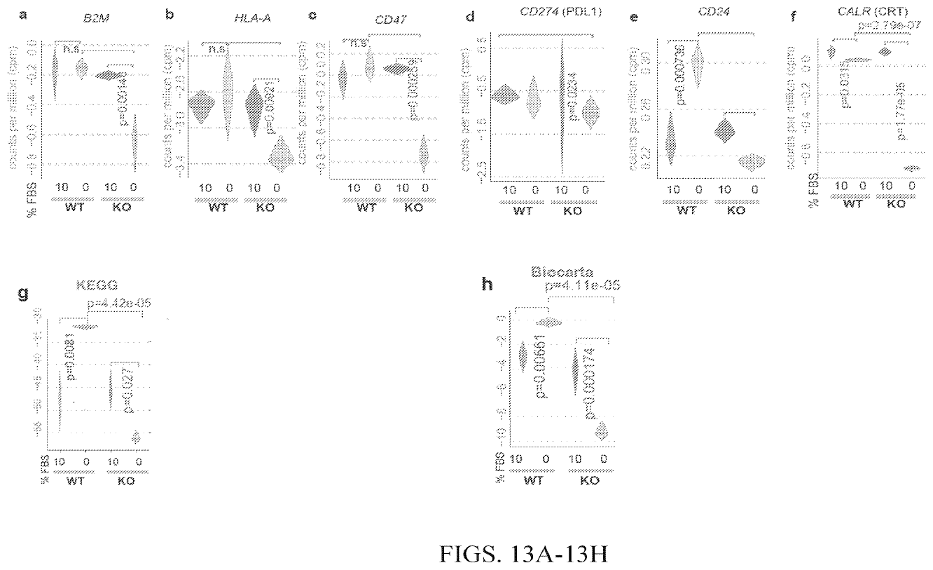

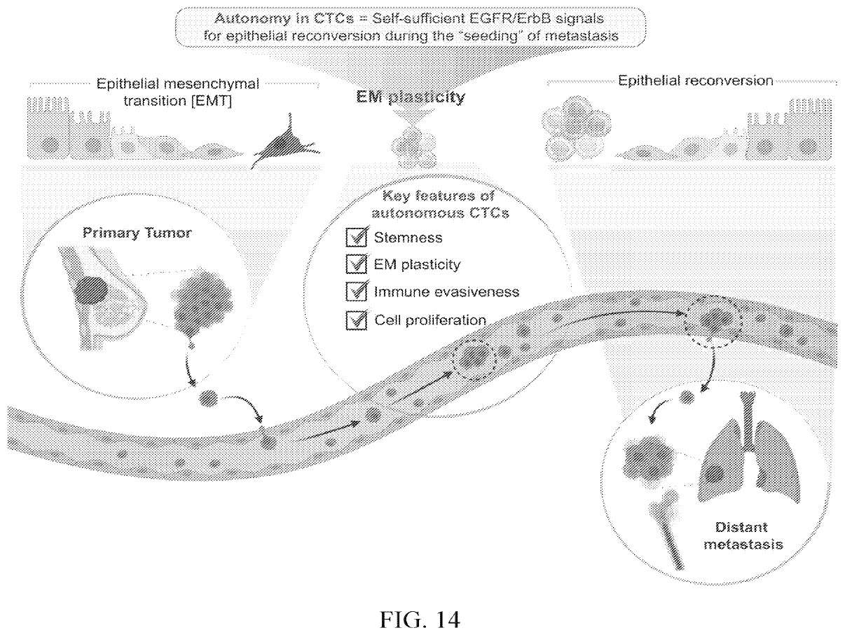

Violin plots display the composite score of the DEGs in a (used as an ‘autonomy signature’) in parental (WT) and GIV-depleted (GIV-KO) MDA MB-231 cells. FIG. 8C. Volcano plot of the differentially expressed genes between WT and GIV-KO MDA MB-321 cells. FIG. 8D. Reactome pathway analyses of the pathways enriched in the genes upregulated in WT vs KO MDA MB-231 cells. Red, pathways associated with EGFR/ERBB signaling. FIG. 8E. Expression of autonomy signature in cells subjected to various stressors is visualized as bubble plots of ROC-AUC values (radius of circles are based on the ROC-AUC) demonstrating the direction of gene regulation (Up, red greyscales; Down, blue greyscales) for the classification of control vs stress conditions. MDA MB-231 cells were used in all except anastasis, which was done in Hela cells. p values based on Welch's T-test (of composite score of gene expression values) are provided either as exact values or using standard code (‘ ’p>0.1, ‘.’ p<=0.1, *p<=0.05, **p<=0.01, ***p<=0.001) next to the ROC-AUC. FIGS. 8F-8H. Schematic (FIG. 8F) of the workflow for proteomic analyses. Briefly, differentially expressed proteins (upregulated in WT, compared to KO; n=3 samples each), as determined by TMT proteomics were used for protein-protein network construction using the human interactome database curated by STRING (Search Tool for the Retrieval of Interacting Genes/Proteins; https://string-db.org/). The resultant network of protein-protein interaction is displayed in FIG. 8G. Node size indicates the degree of connectivity within the network.Degree of connectivity of nodes with a z score of degree (Zd)>=1 is represented as a bar plot in FIG. 8H. FIG. 8I. Reactome pathway analyses of the most connected nodes (Zd>=3) that are upregulated in WT vs KO MDA MB-231 cells.

FIG. 9. Pathway enrichment analysis for differentially upregulated genes in autonomous cells. Differentially expressed proteins (downregulated in WT, compared to KO; n=3 samples each), as determined by RNA sequencing, were used for pathway enrichment analysis using reactome.

FIGS. 10A-10G. Autonomy signature is induced in CTCs, tracks treatment response and carries poor prognosis. FIG. 10A. Left: Schematic displays of the study design for a publicly available dataset, GSE188893, in which the MDA MB-231 breast cancer cell line was injected into NOD scid female mice and then RNA sequencing was carried out for primary tumors from both axillary (P-A) and inguinal (P-I) locations, lymph nodes (L), isolated CTCs, and secondary metastases to the lung (M). Right: Violin plots display the composite score for autonomy signature in all samples and assessed for statistically significant differences compared to the primary tumor by Welch's t-test. FIGS. 10B-10E. Violin plots display the composite score for autonomy signature in various human datasets comparing 15 CTC clusters from single patient during one blood draw against WBC control (FIG. 10B), primary tumors with/without detectable CTCs (FIG. 10C), CTCs challenged or not with hypoxia (FIG. 10D) and single vs. clustered CTCs collected during different times of the day (FIG. 10E). Statistically significance was assessed Welch's t-test. FIG. 10F. Left: Schematic displays study design used in a publicly available dataset, GSE41245. Right: Graph tracks the abundance of CTCs (CTC count; blue greyscales, left y axis), overlaid on composite score of the levels of expression of genes in the autonomy signature in CTCs (EpCAM chip; red greyscales, right y axis). The ratio of epithelial (E) vs mesenchymal (M) markers, as determined by qPCR and reported in the original study by RTqPCR on a panel of markers is indicated. X-axis (time) is annotated with the therapeutic regimen and clinically determined disease status; (P) progression, and (R), recovery. FIG. 10G. Left: Schematic showing the study design in which CTCs were isolated from 31 unique subjects with MBC using CTC-iChip microfluidic device and undergo single-cell CTC RNA-sequence (GSEXXXX). Right: KM curves of overall survival on the same cohort (GSEXXXX), stratified based on high (red greyscales) vs low (green greyscales) autonomy signature. Statistical significance (p value) was determined by the log-rank test.

FIGS. 11A-11K. Autonomy signature is not a mere stress response; it is relatively specific to growth factor restricted conditions. FIGS. 11A-11B. Schematic (left) summarizes the key steps of experimental design to study gene expression during anastasis of HeLa cells that were challenged with 4.3% of EtOH followed by recovery (GSE86480). Violin plot (right) shows Violin plots display the composite score of the genes in the autonomy signature in the HeLa cells, collected at various time points during anastasis. Autonomy signature is significantly suppressed early (within 4 h) during recovery and stays suppressed during late (12 h) recovery. p values based on Welch's T-test (of composite score of gene expression values) are provided besides each time point compared to the initial condition (prior to EtOH application). FIGS. 11C-11I. Violin plots show the composite score of the genes in the autonomy signature in MDA MB-231 cells exposed to different forms of stressful growth conditions. p values based on Welch's T test (of composite score of gene expression values) are provided besides each time point compared to the stress-free (initial/control) condition in each case. FIG. 11J. Venn diagram displays the unique and overlapping genes between the newly identified ‘Autonomy signature’ and five other breast cancer gene signatures with established clinical utility. FIG. 11K. Top: Schematic shows the clinicopathological features of various subtypes of breast cancers: (from left to right, Lum A, Lum B, HER2+, Basal and Claudin-low). Bottom: The composite score of levels of expression of ASig genes (top row) and genes in other clinically useful signatures (rows 2-5) in the various subtypes of breast cancers compared to normal-like breast tissue is visualized as bubble plots of ROC-AUC values (radii of circles are based on the ROC-AUC) in four independent datasets, demonstrating the direction of gene regulation (Up, red greyscales; Down, blue greyscales) for the classification of samples (gene signatures in columns; dataset and sample comparison in rows). The direction of gene expression also indicated the abundance of stemlike/dormant cells in primary tumor. p values based on Welch's T-test (of composite score of gene expression values) are provided using standard code (*p<=0.05, **p<=0.01, ***p<=0.001) next to the ROC-AUC.

FIGS. 12A-12K. Autonomy is associated with reversible EMT, capacity to evade the immune cells and proliferate. FIGS. 12A-12B. Schematic (FIG. 12A) displays the key steps in study design using Her2-transformed mammary epithelial cells (HMI.F, parental). Induction of reversible EMT (by TGFβ) but not stable EMT (by Lapatinib) is associated with the development of long-distance metastases to bones (BM). Parental, reversible EMT, stable mesenchymal and bone metastases-derived clones of HMLE cells were subjected to RNA sequencing. Violin plots (FIG. 12B) display the composite score of the genes in the autonomy signature in the HMLE clones. p values based on Welch's T-test, comparing TGFB-induced reversible EMT and other clones. FIGS. 12C-12H. Schematics summarize the paired CTC-macrophage (FIG. 12C), or CTC-platelets (FIG. 12E) or CTC-NK cell (FIG. 12G) components known to enhance the metastatic potential of heterotypic CTC clusters³⁹. Violin plots (left panels; FIG. 12D, FIG. 12F, FIG. 12H) show the composite score of various markers of immune evasion in CTCs in parental (WT) and GIV-depleted (GIV-KO) MDA MB-231 cells. Heatmaps (right panels; FIG. 12D, FIG. 12F, FIG. 12H) show the expression of each gene. Schematic (FIG. 12I) summarizes the study showing cell cycle activation in CTC-neutrophil clusters. Violin plots show either the composite score of expression of genes in that specific CTC-associated cell cycle signature (FIG. 12J) or levels of expression of MKI67 (FIG. 12K) in parental (WT) and GIV-depleted (GIV-KO) MDA MB-231 cells.

FIGS. 13A-13H. Violin plots show the levels of expression of various genes that encode checkpoint proteins in parental (WT) and GIV-depleted (GIV-KO) MDA MB-231.

FIG. 14. Summary of findings and working model. Schematic showing the epithelial to mesenchymal (FMT) and mesenchymal to epithelial (MET) transition states in primary (left) or metastatic (right) tumors, and the transitioning state in CTC clusters. Growth signaling autonomy, which is seen in CTCs, appears to support a self-sufficient EGFR/ERBB signaling program that is required for re-epithelialization during metastasis. Other properties of autonomous CTCs include, stemness, EM-plasticity, immune evasiveness, and cell proliferation.

### DETAILED DESCRIPTION

Various further aspects and embodiments of the disclosure are provided by the following description. Before further describing various embodiments of the presently disclosed inventive concepts in more detail by way of exemplary description, examples, and results, it is to be understood that the presently disclosed inventive concepts are not limited in application to the details of methods and compositions as set forth in the following description. The presently disclosed inventive concepts are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the presently disclosed inventive concepts may be practiced without these specific details. In other instances, features which are well known to persons of ordinary skill in the art have not been described in detail to avoid unnecessary complication of the description. All of the compositions and methods of production and application and use thereof disclosed herein can be made and executed without undue experimentation in light of the present disclosure.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises,” “comprising.” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a cell, a pharmaceutical composition, and/or a method that “comprises” a list of elements (e.g., components, features, or steps) is not necessarily limited to only those elements (or components or steps), but may include other elements (or components or steps) not expressly listed or inherent to the cell, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

In an aspect, the disclosure provides a method of treating or preventing tumor metastases in a subject in need thereof, comprising administering an effective amount of a treatment for the tumor and/or tumor metastases to the subject. In some embodiments, the cancer is breast cancer.

The terms “subject,” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject,” “patient” or “individual” as used herein, includes any animal that that can be treated by any means known or discovered in the future. Suitable subjects (e.g., patients) include laboratory animals (such as mouse, rat, rabbit, or guinea pig), farm animals, and domestic animals or pets (such as a cat or dog). Non-human primates and, preferably, human patients, are included.

In some embodiments, administering comprises administering a therapeutically effective amount to a subject.

As used herein, the term “amount” refers to “an amount effective” or “an effective amount” of a cell to achieve a beneficial or desired prophylactic or therapeutic result, including clinical results. As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

A model system for studying hallmarks of cancer, particularly growth signaling autonomy in circulating tumor cells for initiating breast cancer metastases, is provided. This model may include evaluating self-sufficiency in growth signaling or growth signaling autonomy by defining the transcriptome, proteome, and phenome of such autonomous state, and unravel its role during cancer progression. The autonomous state is prominently induced in CTCs, which empowers them with key properties that may make CTCs better ‘seeds’ for metastasis.

Self-sufficiency (autonomy) in growth signaling, the earliest recognized hallmark of cancer, is fueled by the tumor cell's ability to ‘secrete-and-sense’ growth factors. This translates into cell survival and proliferation that is self-sustained by auto-secretion and/or paracrine secretion. The breast cancer cells that are either endowed or impaired in growth signaling autonomy demonstrate how autonomy impacts cancer progression.

Autonomy is associated with enhanced molecular programs for stemness, immune evasiveness, proliferation, and epithelial-mesenchymal plasticity (EMP). Autonomy is both necessary and sufficient for anchorage-independent growth factor-restricted proliferation and resistance to anti-cancer drugs and is required for metastatic progression. Transcriptomic and proteomic studies show that autonomy is associated with self-sustained EGFR/ErbB signaling. A gene expression signature (i.e., autonomy signature) was derived which revealed that autonomy is induced in CTCs, and the precursor cells that re-epithelialize initiate metastases. Autonomy in CTCs tracks therapeutic response and prognosticates outcome. Autonomy is preserved during reversible (but not stable) EMT. It is therefore apparent that growth signaling autonomy plays a role in the blood-borne dissemination of human breast cancer.

Disclosed herein is a distinct circulating tumor cell (CTC) phenotype, i.e., growth signaling autonomy that is induced in CTCs, but not in primary tumors or established metastases. Growth signaling autonomy, or self-sufficiency in growth factor signaling, is the first of the six hallmarks of all cancers to be defined²¹, and remains one of the least well understood. Many cancer cells synthesize growth factors to which they are responsive, creating a positive feedback signaling loop called autocrine stimulation²². In fact, serum-free cell culture studies squarely implicate autocrine secretion of growth factors as key support for intracellular mechanisms that impart autonomy²³. Recently, using an integrated systems and experimental approaches, a molecular machinery has been described which is critical for multiscale feedback control to achieve secretion-coupled autonomy in eukaryotic cells²⁴. The machinery is comprised of two species of GTPases, coupled by the multi-modular scaffold GIV, (i.e., Ga-interacting vesicle associated protein; Girdin; gene CCD88A) within a closed-loop circuit that is localized at the Golgi²⁵. Coupling within such a closed-loop control system generates dose-response alignment behavior of sensing and secreting growth factors and is critical for multiscale feedback control to achieve secretion-coupled autonomy²⁶. Consequently, cells with a coupled circuit are self-sufficient in growth signaling, i.e., autonomous, and can survive and achieve homeostasis in serum-restricted conditions; cells in which the circuit is uncoupled (as in GIV-KO cells) are not. Evidence for the requirement of such autonomy in CTCs, and the biological implications and translational potential of these observations, are provided herein.

The autonomous state is prominently induced in CTCs, and empowers them with key properties which may make them better ‘seeds’ for metastasis (FIG. 14).

The autonomous state that is unique to CTCs supports a gene expression program for stemness (i.e., oncogenic de-differentiation), proliferation, immune evasiveness, and epithelial mesenchymal plasticity (EMP). Of these features, the most distinct one is EMP, because, much like what is seen in the case of growth signaling autonomy, EMP is unique to CTCs and very rare in primary tumor cells or in metastases⁴⁰. Autonomy is reduced and/or lost in cells that are unable to transition between E↔M (epithelial and mesenchymal) states. This is consistent with the notion that CTCs are the metastatic precursors that simultaneously express epithelial and mesenchymal markers and best display dynamic E→M and M→E transitions¹⁰. Such a hybrid state in which CTCs have acquired only a partial mesenchymal state allows rapid E→M transitions allow the CTCs to migrate and intravasate, and to quickly reverse M→E to reinitiate a tumor at a distant site⁴¹. Consequently, epithelial-type CTCs with a restricted mesenchymal transition initiate metastases efficiently, whereas mesenchymal-type CTCs do not¹². Consistent with prior findings that CTC clusters express cell-cell adhesion proteins that are components of tight junctions and desmosomes^{16,42}, the autonomous state is endowed with gene expression program to support tight junctions. Divergent from classical epithelial-mesenchymal transition (EMT), EMP is known to also induce unique immunomodulatory effects^{43,44}. Because EMP has been broadly implicated in metastasis, chemoresistance and immunosuppression⁴⁵, and is present in autonomy-endowed cells that outperformed autonomy-impaired cells in their ability to initiate metastases in mice, autonomy-endowed CTCs are likely to be more efficient in seeding metastases than those CTCs that are autonomy-impaired.

Mechanistically, growth signaling autonomy is supported by a secretion-coupled sensing circuit at the Golgi apparatus^{24,25} that controls the secretory flux and organelle shape (i.e., compact stacks when the circuit is enabled, or fragmented stacks when the circuit is disabled). This demonstrates that the CTC population level behavior is dominated by a few high-secreting cells⁴⁶, and that Golgi shape is a strong correlate of CTC-EMT and invasiveness⁴⁷. Although it remains unknown if or how the Golgi-resident circuit begets EMP, growth signaling autonomy and phenotypic plasticity, two hallmarks of cancer, co-exist in CTCs.

Transcriptomic and proteomic analyses pinpointed with surprising convergence that the autonomous state supports a self-sufficient FGFR/ErbB-centric signaling program in the absence of external growth factors. While most cells can be stimulated by growth factors made by neighboring cells-via the process of paracrine signaling—many cancer cells acquire self-sufficiency such that they sense and respond to what they synthesize and secrete, creating a positive feedback sense-and-secrete loop known as autocrine stimulation²². Such type of self-sufficiency or autonomy in a cancer cell obviates its need to depend exclusively on the surroundings, especially during the initial phase of avascular growth of a CTC that has just extravasated to a new site. Although examples of such autonomy exist in the case of PDGF and TGFα by glioblastomas and sarcomas, respectively, autocrine autonomy in the EGF/EGFR pathway has not been described previously and mechanisms that support such sense-and-secrete loops in eukaryotes had remained elusive. By demonstrating that a secretion-coupled-sensing machinery at the Golgi^{24,25} that requires scaffolding by GIV is essential for tumor cells to achieve a state of self-sufficiency in EGFR/FrbB signaling, it is not unusual to find that such a state is endowed with a multitude of pro-oncogenic biological processes that are known to be supported by EGFR/ErbB signals, including cell anchorage-independent cell growth, stemness, EMP and metastasis⁴⁸. Findings are also in keeping with prior observation of EGFR gene induction in EMT that is accompanied by plasticity and tumorigenicity (but not in EMT alone⁴⁹). Although it remains unclear if this is related to the previously reported prognostic roles of high EGFR⁵⁰ and Her2⁵¹ in the serum of patients with breast cancers, EGFR/ErbB2 has been detected in CTCs consistently during serial blood draws⁵² and was found to be activated (as determined by the presence of its phosphorylated state⁵³ in CTCs) with increasing frequency often during MBC progression53. Because inhibition of EGFR prevents CTC clustering and diminishes its metastatic potential⁵⁴, it is possible that higher autonomy observed in the CTC-clusters (compared to single CTCs) requires the autonomous signaling machinery (the sense-and-secrete loop) for CTCs to remain clustered and maintain its metastatic potential. It is possible that the EGF-predominant autocrine loop maintains CTC-junctions by triggering both the secretion of junctional proteins and/or complexes (e.g., E-cadherin) from the Golgi to the plasma membrane (PM)⁵⁵ and their subsequent activation at the junctions⁵⁶.

A gene signature for growth signaling autonomy can track treatment response in a single index patient, and more importantly, the signature prognosticates outcome. Molecular markers of the metastatic potential of CTCs, such as RPL15⁷ and a 17-gene signature⁵⁷, have been described in other works. While RPL15 was identified during an in vivo genome-wide CRISPR activation screen to identify genes in breast cancer patient-derived CTCs that promote their distant metastasis in mice, the latter sequence was trained on a cohort of normal vs. primary tumor and whole blood from patients. The present disclosure not only adds growth signaling autonomy to the growing list of the parameters that help define the ‘fitness’ of CTCs, but also provides a methodology to objectively measure (using a gene signature) the degree of autonomy (and hence, the fitness) of CTCs to serve as ‘seeds’ for metastases.

Thus, the present disclosure provides insights into what determines the success of a CTC to serve as metastatic “seed”. By demonstrating that the detached tumor cells (sans ECM contact) gain autocrine self-sufficiency in growth factor signaling and phenotypic plasticity in circulation, while maintaining the properties of stemness, proliferation and immune evasiveness (which are seen also in primary tumors), it is shown that the coexistence of two hallmarks of cancer that are relatively unique to CTCs and are intricately intertwined.

As applied to clinical use, RNA sequencing and/or gene extraction may be beneficial in determining the metastatic potential of CTCs present in patient tumors. This may be achieved by extracting RNA from blood, however this may be difficult due to the limited quantity of cells in blood samples and/or the efficiency of RNA extraction from blood samples, so alternative methods for determining metastatic potential of CTCs may be required. For example, in some embodiments, the gene signature for the metastatic potential of CTCs in a patient is identified using fluorescent in situ hybridization (FISH). FISH may be used to detect mRNA in situ on CTC preps from blood. In other embodiments, NanoString technology is used to determine the metastatic potential of CTCs. NanoString technology may be used with a small number of target sequences that are rank ordered by rigorous statistical cutoffs, such that the NanoString low RNA input nCounter assay may provide reliable gene expression profiling of CTCs. A NanoVelcro CTC-RNA assay may therefore include the Thermoresponsive (TR)-Nano Velcro CTC purification system and the NanoString nCounter.

### EXAMPLES

### Study Design

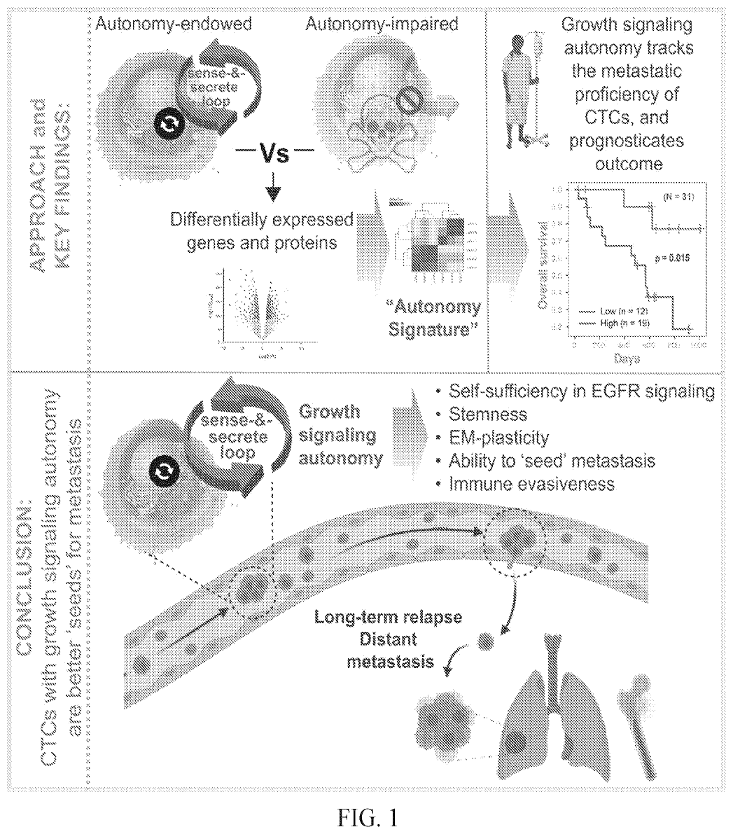

To study how autonomy in cancer cells impact cancer progression, and more specifically, breast cancers, two MDA MB-231 breast cancer cell lines were used, that are either endowed (WT) or impaired (GIV-KO by CRISPR²⁴) in growth signaling autonomy (FIG. 1). These cells were used because they are highly aggressive, invasive, and poorly differentiated triple-negative breast cancer (TNBC) cell line which lacks the estrogen receptor (ER), progesterone receptor (PR), as well as the HER2 (human epidermal growth factor receptor 2) receptor, which is one of the most widely used triple-negative cell lines in metastatic breast cancer research²⁷. It is also a cell line that has been shown to require GIV for growth signaling autonomy²⁴. These cells were analyzed by functional and ‘omics’-based approaches to navigate the uncharted territory of cancer cell autonomy. Because the GTPase circuit for autonomy requires GIV's modules/motifs that evolved only in the higher eukaryotes²⁴, and GIV is overexpressed in most cancers²⁸, it was hypothesized that tumor cells may frequently assemble and utilize such evolutionary vantage to achieve growth signaling autonomy at some stage during cancer progression. Using an integrated computational and experimental approach, the pair of cell lines were systematically analyzed for key hallmarks of cancer cells.

### Cell Lines

MDA-MB-231 cells were grown at 37° C. in their suitable media, according to their supplier instructions, supplemented with 10% FBS, 100 U/ml penicillin, 100 μg/ml streptomycin, 1% L-glutamine, and 5% CO2. GIV knock-out (KO) cell lines were generated using Pooled guide RNA plasmids (commercially obtained from Santa Cruz Biotechnology; Cat #sc-402236-KO-2), as described earlier⁵⁸. Briefly, these CRISPR/Cas9 KO plasmids consist of GFP and Girdin-specific 20 nt guide RNA sequences derived from the GeCKO (v2) library and target human Girdin exons 6 and 7. Plasmids were transfected into Hela and MDA-MB-231 cells using PEI. Cells were sorted into individual wells using a cell sorter based on GFP expression. To identify cell clones harboring mutations in gene coding sequence, genomic DNA was extracted using 50 mM NaOH and boiling at 95° C. for 60 mins. After extraction, pH was neutralized by the addition of 10 % volume 1.0 M Tris-pH 8.0. The crude genomic extract was then used in PCR reactions with primers flanking the targeted site. Amplicons were analyzed for insertions/deletions (indels) using a TBE-PAGE gel. Indel sequence was determined by cloning amplicons into a TOPO-TA cloning vector (Invitrogen) following the manufacturer's protocol. These cell lines were characterized in earlier work^{24,58}, and modified here for luciferase expression.

MCF7 cells were purchased from the ATCC and verified cells by STR profiling through the University of Michigan Advanced Genomics Core. MCF7 cells were cultured in DMEM medium with 10% serum, 1% penicillin/streptomycin, and 1% glutamax (ThermoFisher Scientific) in an incubator set at 37° C. and 5% CO2. Click beetle green luciferase (CBG) was expressed in these cells by lentiviral transduction as described previously⁵⁹. To stably express full-length, wild-type GIV in MCF7 and MDA-MB-231-GIV KO cells, a Tol2 transposon system was used. The Tol2 transposon vector uses a CAG promoter to drive constitutive expression of the cDNA for CCDC88A (the gene name for GIV) and a hygromycin resistance gene linked by a P2A sequence (Vector Builder). Cells were co-transfected with the Tol2 GIV transposon and a Tol2 transposase (Vector Builder) at a 3:1 ratio of micrograms of plasmid DNA using Fugene 6 (Promega) according to the manufacturer's directions. Control cells underwent transfection with an empty Tol2 transposon and Tol2 transposase at the same ratio of plasmid DNA. Two days after transfection, hygromycin (Sigma-Aldrich) was added to select batch populations of cells stably expressing GIV. After obtaining stable cell lines, resultant MCF7-GIV cells were not cultured in hygromycin.

### Immunoblotting

To verify the expression of GIV in MCF7-GIV and MDA-MB-231-GIV KO/WT cells, equal aliquots of whole-cell lysates (prepared using RIPA buffer) were loaded on a 8% SDS PAGE gel and immunoblotting was carried out for GIV with the mouse monoclonal antibody H-6 (Santa Cruz Biotechnology) as described previously⁶⁰. Immunoblots were analyzed using LI-COR Odyssey® Imaging System (LiCOR bio, Lincoln, NE) and the recommended companion software, Image Studio.

### RNA Sequencing and Identification of DEGs

WT and GIV-KO MDA-MB231 cells grown in 0% and 10% serum concentration in p10 dishes (Corning) for 16 h prior to harvest, and cell pellets were subsequently processed for RNA extraction using a kit (R2052, Zymo Research) as per manufacturer's protocol. Isolated RNA has been processed for RNA sequencing in the Illumina NovaSeq 6000 platform. Fastq sequence files have been mapped using the human GRCh38 genome.

For HeLa cells, RNA sequence studies were conducted using the same approach as above, with two differences.

For MCF7 cells, cells were grown either in low glucose, low serum, estrogen-supplemented phenol red free DMEM (4 mM glucose, 1% serum, 1% glutamax, and 10 nM estrogen in DMEM with no glucose or phenol red (Gibco, catalog number A1443001)) or in high glucose, phenol red free DMEM without (25 mM glucose, 0% serum, no estrogen). Cells were cultured in low glucose, low serum, and estrogen-supplemented medium for 3 days or high glucose, serum-free, and estrogen-free DMEM for 1 day before collecting cancer cells for RNA.

### Tandem Mass Tag™ (TMT) Proteomics

WT and GIV-KO MDA-MB231 cells were maintained in 0% and 10% serum concentration in p10 dishes (Corning) for 16 h prior to harvest, and cell pellets were subsequently processed for TMT proteomics using LUMOS Orbitrap-Fusion analyzer. Peptides are identified and mapped using Peaks X Pro pipeline. The intensity ratio of each identified protein in WT MDA-MB231 Vs GIV-KO MDA-MB231 cells was identified and selected if the significance score>20.

### Soft Agar Growth Assays

Assays were performed to assess the viability of cell cultures⁶. A base layer of 0.7% agar (Sigma-Aldrich) dissolved in DMEM medium was made, adding 0.2% serum after the agar solution cooled to ˜37° C. before transferring 1 ml per well to 6 well plates. After the base layer solidified at room temperature, 0.35% low melting agar (Sigma) in DMEM medium was prepared, adding the same concentration of serum as in the base layer and 5×10⁴ cells per ml when the solution cooled to ˜37° C. (n=3 wells per cell type and serum condition). 1 ml per well of the low melting point agar/cell solution was immediately transferred to each well and cooled the plate briefly at 4° C. before placing in a cell culture incubator. 0.5 ml fresh DMEM medium with 0.2% serum was added every 2 days. After one week, 9 bright-field images per well were obtained on an inverted microscope (Olympus IX73 with 20× objective). Immediately after microscopy, 150 μg/ml luciferin (Promega) was added to each well, incubated in a cell culture incubator for 10 minutes, and then acquired a bioluminescence image of total viable cells on an IVIS Lumina (30 second image, large field of view) (Perkin Elmer). A person blinded to experimental conditions enumerated colonies and quantified imaging data (Living Image, Perkin Elmer).

### Cytotoxicity Assays

Cytotoxicity assays were performed on MCF7 cells as described previously⁶². Briefly, 7.5×10³ MCF7 WT or MCF7-GIV cells per well were seeded in black wall 96 well plates (ThermoFisher Scientific, catalog number 165305). One day after seeding in normal growth medium, cells were washed once with PBS and then various concentrations of drugs (tamoxifen, fulvenstrant, or palbociclib; all purchased from Tocris) were added in phenol red free DMEM (ThermoFisher Scientific) with 4 mM glucose, 1% serum, 1% penicillin/streptomycin, 1% Glutamax, and 10 nM estrogen (n=4 wells per cell type and concentration). Three days later, μg/ml luciferin per well was added, incubated in a cell culture incubator for 10 minutes, and then acquired a bioluminescence image of total viable cells on an IVIS Lumina (1 minute image, large field of view). Bioluminescence was quantified as radiance per well (LivingImage software) and normalized data for each cell type and drug concentration to vehicle only.

### Fluorescence Lifetime Imaging Microscopy (FLIM) of NADH

1.5×10⁵ cells were seeded in complete DMEM medium in 35 mm dishes. One day later, cells were washed once with PBS and then 2 ml per dish of Fluorobrite DMEM medium (ThermoFisher) was added with 10% serum and either 0-mM or 0.5-mM glucose. After incubation overnight, cells were imaged with an Olympus FVMPE-RS upright microscope with Olympus Fluoview software, Spectra-Physics Insight DeepSee laser, and ISS Fast FLIM. For NADH FLIM imaging, 740 nm excitation was used and captured the endogenous NADH lifetime in the blue channel with a bandpass filter of 410-460 nm using ISS FastFLIM software. FLIM data was acquired and analyzed⁶³. Briefly, raw data was exported from ISS VistaVision and used custom MATLAB scripts to segment and quantify images.

### Animal Studies

Prior to mouse experiments, MDA-MB-231 WT and MDA-MB-231-GIV KO/WT cells were cultured in serum-free DMEM with 25 mM glucose overnight. It was verified that these cells did not lose viability after overnight culture in serum-free medium relative to medium with 10% serum as determined by cell-based measurements of CBG bioluminescence in an IVIS Lumina (Perkin Elmer). 1×10⁵ breast cancer cells were injected per mouse (n=5 per each cell type), verifying positioning of the 30 g needle in the left ventricle by return of pulsatile bright red blood⁶⁴. Bioluminescence was imaged with an IVIS Spectrum (Perkin Elmer) in mice at various time points and data was quantified with LivingImage software. For each mouse, the fold-change in bioluminescence was calculated relative to the value obtained one day after injection to normalize for variations in injected amounts of cells. The area-under-the-curve±SEM was calculated for total bioluminescence in each group.

### Transcriptomic Datasets

All publicly available transcriptomic datasets were downloaded from National Center for Biotechnology Information (NCBI) Gene Expression Omnibus website (GEO) 65-67 or European Molecular Biology Laboratory (EMBL) European Bioinformatics Institute (EMBL-EBI) ArrayExpress website⁶⁸. All gene expression datasets were processed separately using the Hegemon (hierarchical exploration of gene expression microarrays on-line) data analysis framework⁶⁹⁻⁷¹. Datasets that belonged to two different platforms were not combined.

### StepMiner Analysis

StepMiner is an algorithm that identifies step-wise transitions using step function in a time-series data⁷². StepMiner undergoes an adaptive regression scheme to verify the best possible up and down steps based on sum-of-square errors. The steps are placed between time points at the sharpest change between expression levels, which provides information about timing of the gene expression-switching event. To fit a step function, the algorithm evaluates all possible steps for each position and computes the average of the values on both sides of a step for the constant segments. An adaptive regression scheme is used that chooses the step positions that minimize the square error with the fitted data. Finally, a regression test statistic is computed as follows:

$$F stat = \frac{{\sum }_{i = 1}^{n} {( {\hat{X}}_{l} - \overline{X} )}^{2} / ( m - 1 )}{{\sum }_{i = 1}^{n} {( {X}_{i} - {\hat{X}}_{l} )}^{2} / ( n - m )}$$

Where Xᵢ for i=1 to n are the values, \hat{X}ₗ for i=1 to n are fitted values. m is the degrees of freedom used for the adaptive regression analysis. X̅ is the average of all the values:

$$\overline{X} = \frac{1}{n} * {\sum }_{j = 1}^{n} {X}_{j} .$$

For a step position at k, the fitted values \hat{X}ₗ are computed using

$$\frac{1}{k} * {\sum }_{j = 1}^{n} {X}_{j} for i = 1 to k and \frac{1}{( n - k )} * {\sum }_{j = k + 1}^{n} {X}_{j} for i = k + 1 to n .$$

### Composite Gene Signature Analysis Using Boolean Network Explorer (BoNE)

Boolean network explorer (BoNE) provides an integrated platform for the construction, visualization and querying of a gene expression signature underlying a disease or a biological process in three steps. First, the expression levels of all genes in these datasets were converted to binary values (high or low) using the StepMiner algorithm. Second, Gene expression values were normalized according to a modified Z-score approach centered around the StepMiner threshold (formula=(expr−SThr)/3*stddev). Third, the normalized expression values for every genes were added together to create the final score for the gene signature. The samples were ordered based on the final signature score. Classification of sample categories using this ordering is measured by ROC-AUC (Receiver Operating Characteristics Area Under the Curve) values. Welch's Two Sample t-test (unpaired, unequal variance (equal_var=False), and unequal sample size) parameters were used to compare the differential signature score in different sample categories. Violin, Swarm and Bubble plots are created using Python Seaborn Package version 0.10.1. Pathway enrichment analyses for genes were carried out via the reactome database and algorithm⁷³. Violin, Swarm and Bubble plots are created using Python's Seaborn Package version 0.10.1.

### Survival Outcome Analysis

Kaplan-Meier (KM) analyses were done for different gene signatures. The high and low groups were separated based on the StepMiner threshold on the composite score of the gene expression values. The statistical significance of KM plots was assessed by log rank test. Kaplan-Meier analyses were performed using Python's Lifeline Package version 0.14.6.

### Protein-Protein Interaction

Upregulated proteins in WT MDA-MB-231 cells in comparison with the GIV-KO MDA-MB-231 cells are identified with an intensity ratio cutoff of >=2 and with a significance value>=20. Interaction edges between the identified proteins are fetched from the STRING human interactome database and represented in FIG. 10G as a protein-protein interaction network (PPIN) using Gephi 9.02.Degree distribution of the PPIN computed using Python Network Package.

### Quantification and Statistical Analysis

Gene signature is used to classify sample categories and the performance of the multi-class classification is measured by ROC-AUC (Receiver Operating Characteristics Area Under the Curve) values. A color-coded bar plot is combined with a density plot to visualize the gene signature-based classification. All statistical tests were performed using R version 3.2.3 (2015 Dec. 10). Standard t-tests were performed using Python scipy.stats.ttest_ind package (version 0.19.0) with Welch's Two Sample t-test (unpaired, unequalvariance (equal_var-False), and unequal sample size) parameters. Multiple hypothesis correction was performed by adjusting p values with statsmodels.stats.multitest.multipletests (fdr_bh: Benjamini/Hochberg principles). Sample number of each analysis is provided with associated plots beside each GSE ID number or sample name.

The statistical significance of KM plots was assessed by log rank test. Pathway enrichment analyses of gene lists were carried out using the Reactome database⁷⁴ and the Cytoscape plug-in, CluGo.

### Growth Signaling Autonomy is Associated with Stemness and Epithelial-Mesenchymal Plasticity

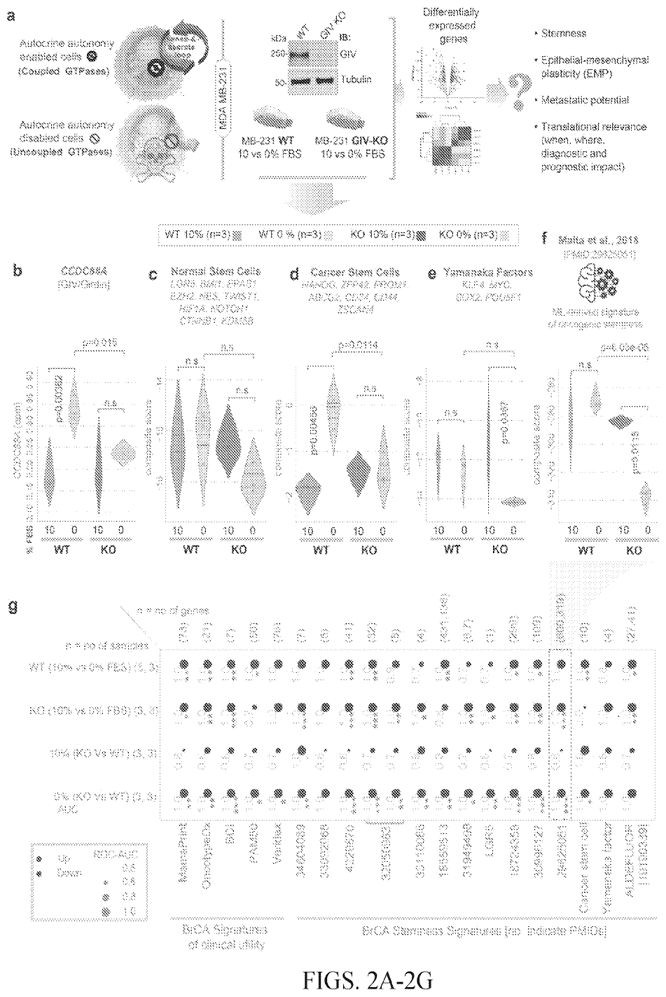

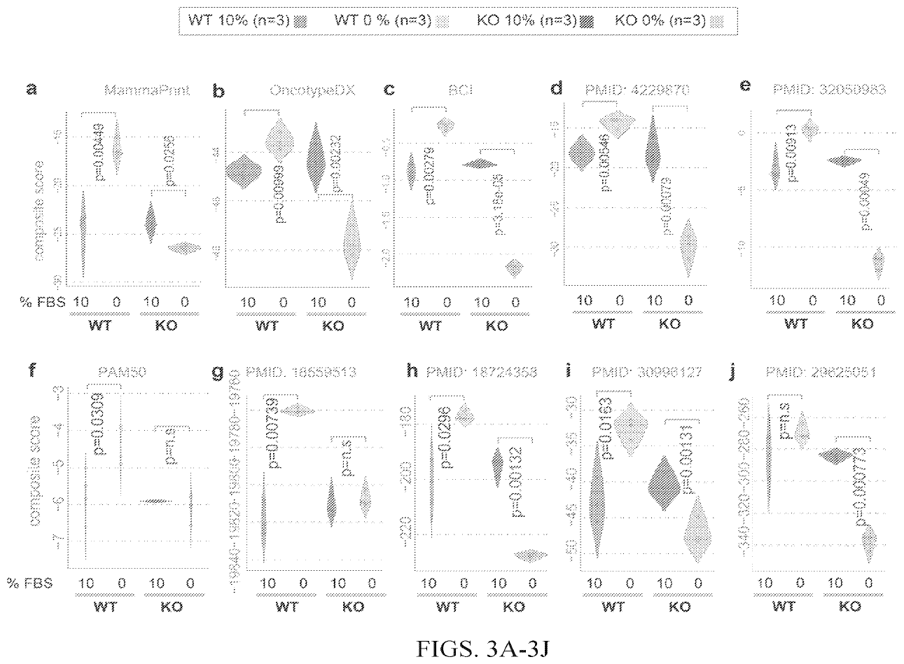

The expression of (CDC88A gene (which encodes GIV) was significantly upregulated when the autonomous WT, but not GIV-KO cells were switched from 10% to 0% serum conditions (FIG. 2B), which is consistent with the increased need for autocrine growth factor signaling during serum starvation. Although conventional markers of normal pluripotent stem cells remained unchanged during serum depravation in both cells (FIG. 2C), markers of cancer stem cells (CSCs) followed the same pattern as (CDC88A (FIG. 2D). When the core pluripotency master regulators (the Yamanaka factors²⁹) (FIG. 2E) and the breast cancer-specific indices of the degree of oncogenic dedifferentiation (identified using a machine learning algorithm³⁰) (FIG. 1F) were analyzed, the autonomous WT cells maintained these signatures despite serum depravation while GIV-KO cells did not. These patterns (induction or maintenance in the autonomous WT, but suppression in GIV-KO cells) were observed repeatedly across a comprehensive panel of gene signatures of breast cancer aggressiveness and stemness that have been reported in the literature (FIG. 2G; FIGS. 3A-3J).

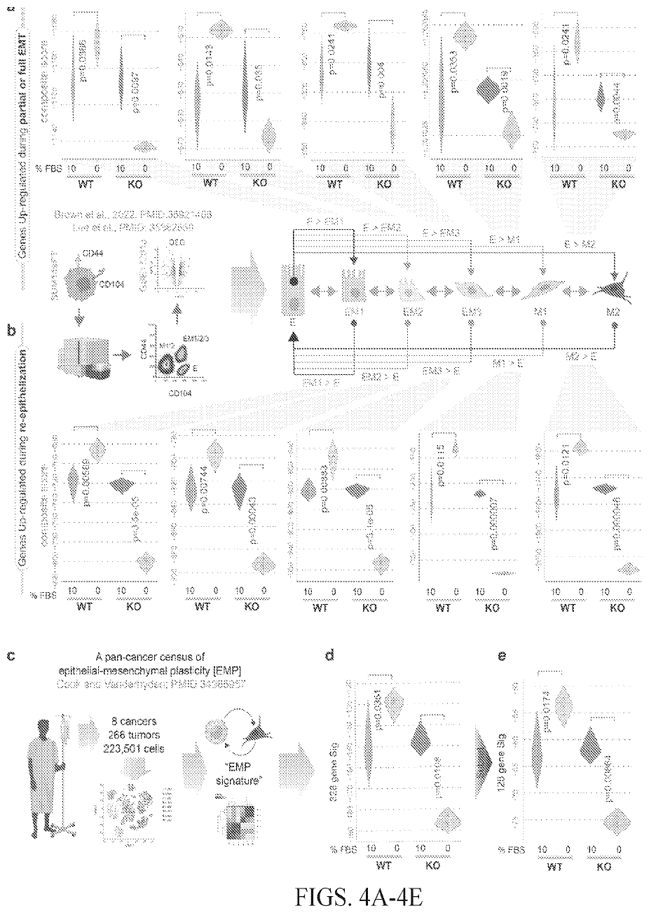

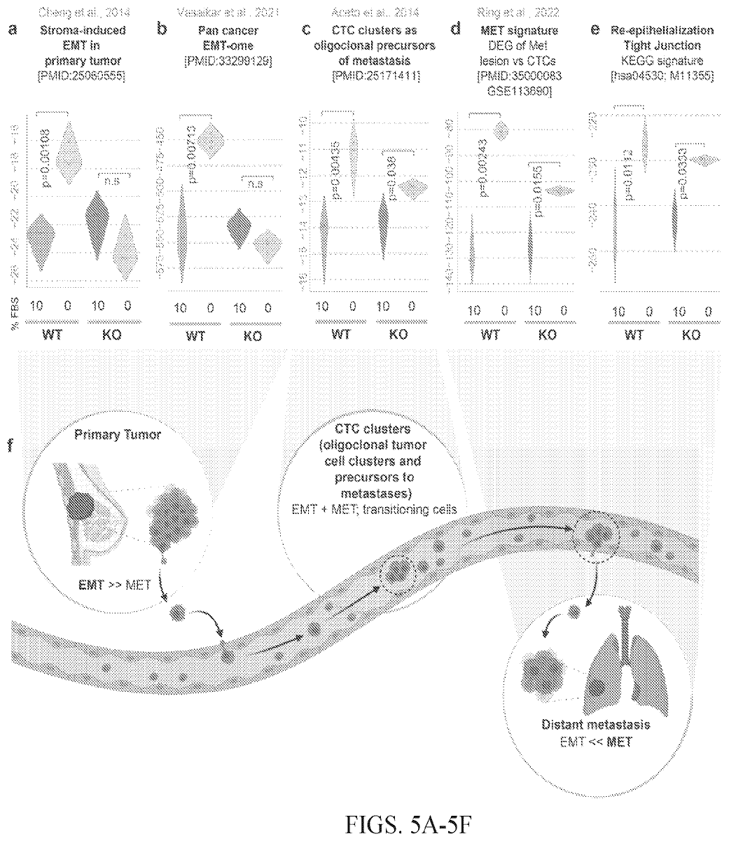

Autonomous WT, but not GIV-KO cells also induced gene signatures epithelial-mesenchymal plasticity (FMP31), i.e., the ability of cells to interconvert between epithelial and mesenchymal phenotypes in response to signals (FIG. 4). For example, all gene signatures derived from isolated distinct single-cell clones from the SUM149PT human breast cell line spanning the E↔EM1-3↔M1-2 spectrum, previously characterized for diverse migratory, tumor-initiating, and metastatic qualities³², were induced in the autonomous WT, but suppressed in the GIV-KO cells during serum depravation (FIGS. 4A-4B). An identical pattern was seen also for the transcriptional census of EMP in human cancers, which was derived by leveraging single-cell RNA sequence data from 266 tumors spanning 8 different cancer types³³ (FIG. 4C). This held true for both the 328-gene EMP consensus signature (FIG. 4D), as well as its cancer cell-specific 128-gene subset (FIG. 4E). Furthermore, numerous gene signatures across the EMT and MET spectrum, derived from diverse human samples representing the stages of metastases (primary tumors, CTCs, and metastases) and genes that are essential for establishing cell-cell junctions were induced in the autonomous WT, but remained unchanged or were suppressed in the GIV-KO cells (FIGS. 5A-5J).

These findings indicate that GIV-dependent growth signaling autonomy is required to support molecular programs of stemness and EMP in growth factor-restricted conditions (i.e., 0% FBS); however, such autonomy is largely dispensable in the presence of excess growth factors because all readouts were indistinguishable when WT and KO cells were compared at 10% FBS (FIGS. 2A-3J).

### Autonomy is Required and Sufficient for Anchorage-Independent Growth, and is Required for Metastatic Spread

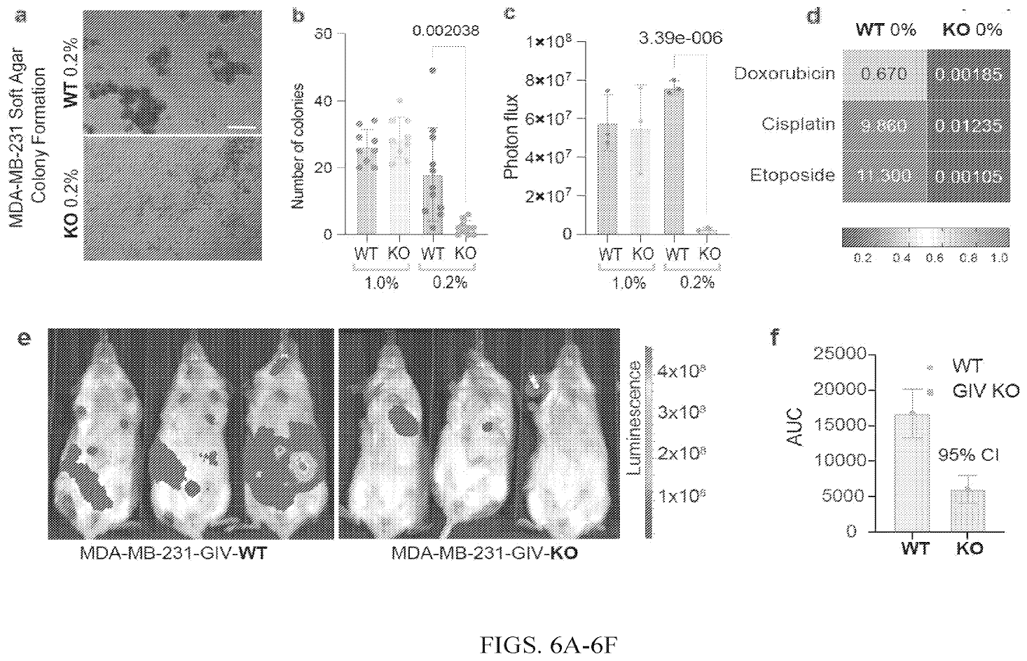

It was next assessed whether the autonomy-endowed and autonomy-impaired cells are capable of anchorage-independent growth, which is a hallmark of anoikis resistance and the path to further steps in metastasis³⁴. When tested for growth as spheroids in soft agar at varying serum concentrations, while both WT and GIV-KO cells did so in the presence of excess serum, only the autonomous WT cells thrived in serum-restricted conditions (0.2% FBS) (FIGS. 6A-6C). Under serum-restricted growth conditions, the autonomous WT, but not the autonomy-impaired GIV-KO cells were also relatively resistant to various conventional chemotherapeutic agents used to treat TNBCs, as determined by the observed differences in their half-maximal inhibitory concentrations (IC50) (FIG. 6D) and displayed significantly higher metastatic potential after intracardiac injection (FIG. 6E-6F). These findings indicate that GIV is required for 3D growth, chemoresistance and metastasis in serum-restricted conditions and that GIV-dependent growth signaling autonomy may be required for these phenotypes.

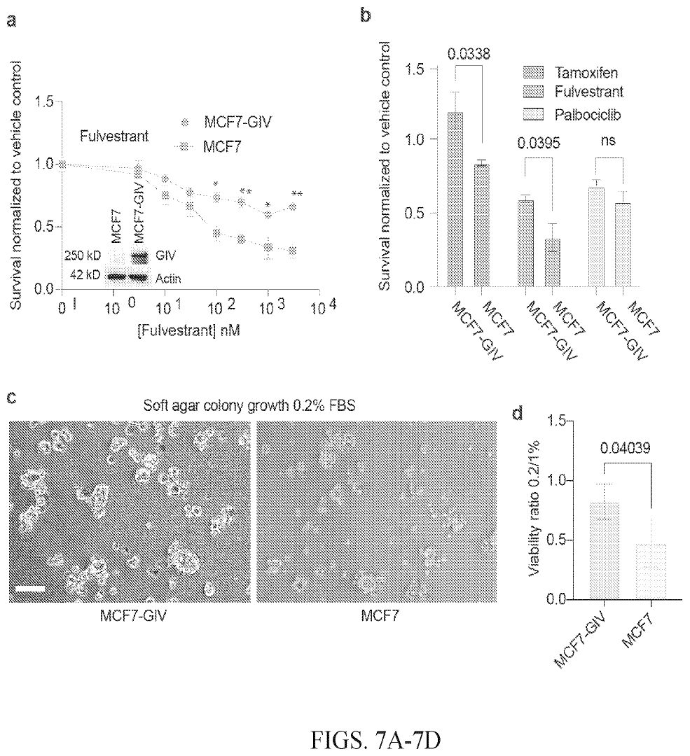

Previous work has documented the absence of full-length GIV in the most widely used non-metastatic ER-positive MCF7 breast cancer cells (43.6% of total PubMed citations²⁷). These MCF7 cells depend on the growth hormone estrogen to proliferate. It was found that restoring GIV expression in these cells using a Tol2-based destination vector was sufficient to enable estrogen-independent growth, as determined using the ER-antagonist Fulvestrant (FIG. 7A-7B) and Tamoxifen) (FIG. 7B). GIV was also sufficient for the growth of MCF7 as spheroids in soft agar under estrogen-and serum-restricted conditions (0.2%) (FIGS. 7C-7D). This impact of GIV on cell growth/survival in serum-restricted conditions was limited to growth factors and hormone, but not for the CDK4/6 inhibitor, Palbociclib (FIG. 7B), a commonly used therapeutic agent in ER+breast cancers. which blocks the cell cycle transition from G1 to S by inhibiting the kinase activity of the CDK/cyclin complex. Findings suggest that GIV-dependent growth signaling autonomy may be sufficient for growth factor and hormone-restricted growth.

### ‘Autonomy’ Represents a Distinct Cell State that is Self-Sufficient in EGER ErbB Growth Signaling

RNA sequencing studies revealed a set of 30 genes (27 upregulated and 3 downregulated) and two miRNAs were most differentially expressed (DEGs; Log fold change>5) (FIG. 8A) between the autonomous WT and the GIV-KO cells. These genes were upregulated and downregulated in WT and KO cells, respectively, in response to serum depravation (FIG. 8B), a pattern that was strikingly similar to those observed previously for signatures of stemness (FIG. 2G) and EMP (FIGS. 4A-4E). No genes were significantly differentially expressed between the two cell lines when cultured in 10% serum. The list of upregulated DEGs was notable for the presence of EGF (FIG. 8C). A reactome pathway analysis confirmed that this list was significantly enriched in genes that participated in the EGFR/ErbB-signaling pathway (FIG. 8D). Pathway analysis of the downregulated DEGs was notable for cellular processes related to the extracellular matrix (ECM), e.g., collagen formation, assembly, and degradation, activation of metalloproteinases and degradation of ECM (FIG. 9).

Using the composite score of expression of the 30 genes as a signature of growth

signaling autonomy (“autonomy signature”), a wide range of cellular stress response states were navigated to assess specificity to growth-factor depravation (as opposed to a non-specific ‘stress response’). The autonomy signature was induced in MDA MB-231 cells challenged with serum depravation (FIG. 11C) but not glucose depravation, oxidative stress, hypoxia, ER-stress, or mechanical compression (FIG. 8E; FIGS. 11C-11I). The signature was suppressed in HeLa cells undergoing anastasis, a process of resurrection in which cancer cells can revive after ethanol-induced apoptosis (FIGS. 11A-11B; FIG. 8E). Furthermore, the autonomy signature had no overlaps with other signatures that are either approved for clinical use by the FDA or in clinical trial for their unitality in the management of breast cancers (FIG. 11J). Together, these findings indicate that the gene expression patters of growth signaling autonomy are unique; they represent a distinct ‘cellular state’ that is induced under serum-restricted conditions and requires GIV.

Tandem Mass Tag (TMT)-based quantitative proteomics studies (FIG. 8F) revealed that that autonomous WT cells differential express a distinct set of proteins, which included EGFR (2.308-fold). When constructing a protein-protein interaction (PPI) network using this list of upregulated proteins fetched from the human interactome, EGFR emerged as the node with highest degree of connectivity (FIGS. 8G-8H). The most connected proteins (i.e., nodes of the PPI network with Z_{d}>=3) that are upregulated in autonomous WT cells was, once again, found to be significantly enriched in proteins that participate in the EGFR/ErbB2 signaling pathway (FIG. 8I).

Thus, the transcriptomic and proteomic studies agree; both reveal an upregulated EGFR/ErbB2 signaling pathway in the autonomous WT cells during serum-restricted conditions and confirm the requirement of GIV in such upregulation. Intriguingly, both EGF gene and EGFR protein emerged from these ‘omics’ studies, with an enrichment of its immediate downstream signaling (e.g., signaling via Grb2, PLCy, CDC42, Rho and Rac GTPases), and both secretory and endocytic trafficking proteins (e.g., COP1, RABs, SNARE, EXOCI ARF6, AP2-subunits, CAV1 proteins) (FIG. 8H). Findings show that the transcriptome and proteome of growth signaling autonomy supports both auto-secretion and/or paracrine secretion and signaling within the EGFR/ErbB pathway.

### Autonomy Signature is Induced in CTCs with High Metastatic Proficiency

Using the autonomy signature as a computational tool, the various steps within the cancer initiation and progression cascade were analyzed. A microarray dataset generated using xenografts of MDA MB-231 cells implanted into inguinal and axillary fat pads of NOD scid female mice, which included samples representing all major steps of the cascade was prioritized (FIG. 10A). Notably, the autonomy signature was neither induced in primary tumors, nor in metastases; it was induced exclusively in circulating tumor cells (CTC) isolated from blood samples (FIG. 10A). Findings in mice were conserved in humans; the autonomy signature was induced in human CTCs (FIG. 10B) but not in human primary tumors when compared to normal, across all molecular subtypes (FIG. 11K). When primary tumors from patients with detectable CTCs were compared to those without, the autonomy signature was higher in tumors that shed CTCs (FIG. 10C).

Next, diverse human CTC datasets that were generated by independent groups were reviewed, in which the metastatic proficiency of the CTCs was experimentally validated using xenograft models. For example, intratumoral hypoxia is a known driver of intravasation of clustered CTCs with high metastatic proficiencyl⁸. It was found that the autonomy signature was significantly higher in hypoxic clusters of live CTCs when compared against their normoxic counterparts (FIG. 10D), all drawn from a breast cancer patient and labeled with HypoxiaRed, a cell-permeable dye that tags hypoxic cells based on their nitroreductase activityl⁸. The shedding of CTCs is known to peak at the onset of night³⁵, when they display more metastatic proficiencyl⁹.

It is also known that compared to single CTCs, the metastatic proficiency of CTC clusters¹⁶ and CTC-WBC clusters⁸ are higher. The autonomy signature was induced in CTC/CTC-WBC clusters, as compared to single CTCs (FIG. 10E). The significance of such induction was higher in CTC-WBC clusters that were collected at night (FIG. 10E).

### Autonomy Signature in (TC's Tracks Therapeutic Response and Prognosticates Outcome

CTCs exhibit dynamic changes in abundance and epithelial and mesenchymal composition during treatment¹⁰, and it was considered if and/or how treatment might impact autonomy signature. A dataset comprised of CTCs serially collected from an index patient was analyzed (FIG. 10F), which displayed reversible shifts between these compositions accompanying each cycle of response to therapy (R) and disease progression (P). The autonomy signature was rapidly downregulated (alongside CTC count) during treatment initiation, which coincided with therapeutic response (1-3 months) (FIG. 10F). The signature was subsequently induced from the third to seventh month, despite continuation of treatment and low CTC counts, and preceded clinically confirmed disease progression at the eighth month which necessitated salvage chemotherapy (FIG. 10F). The signature did not show any discernible relationship with the relative amounts of E/M compositions, which is in keeping with prior observations that autonomy is associated with EM-plasticity (FIG. 4).

A single-cell RNA sequence dataset was also re-analyzed, where the dataset was generated on viable CTCs from 31 unique patients with hormone receptor positive MBC for whom follow-up and outcome data were available (overall survival⁷). CTCs were freshly isolated directly from whole blood using a CTC-iChip microfluidic device³⁶ (FIG. 10G). For those patients who had multiple blood draws on the same day, the last draw of the day was selected. A Kaplan-Meier survival analysis revealed a higher 3-year mortality risk among those with high autonomy signature in CTCs compared to those with low expression of the same (p=0.015) (FIG. 10G).

### Autonomy is Associated with the Potential to Re-Epithelialize, Evade the Immune System, and Proliferate

CTCs must display plasticity between epithelial and mesenchymal states to complete the metastatic process¹⁰, and the EGF/EGFR pathway has been identified as 1 of the 14 major pathways that support EM-plasticity³³. Whether the self-sustained EGF/EGFR signaling program in autonomy is specifically associated with ‘reversibility’ of the EMT process was considered. A dataset in which Her2-transformed human mammary epithelial (HMLE) cells were either programmed for reversible EMT (induced by TGFβ) or to a stable mesenchymal phenotype (by chronic exposure to the ErbB inhibitor, lapatinib) was used (FIG. 12A). Xenograft studies using these programmed cells had confirmed that reversible, but not stable mesenchymal phenotype produces long-bone metastases³⁷. The autonomy signature was found to be higher in cells programmed for reversible EMT compared to both stable mesenchymal cells and established bone metastases (FIG. 12B). This indicates that the autonomous state is present in transformed cells that carry the potential to undergo dynamic transitions (EM-plasticity) but is lost when cells get stuck in either stable mesenchymal (as during the emergence of resistance to Lapatinib) or re-epithelialized states (as in established metastatic colonies).

EMT and stemness in tumor cells correlate with immune checkpoint expression and complex interactions with platelet and immune cells^{30,38}. Similarly, the metastatic proficiency of the CTCs is influenced by complex interactions with the platelets and immune cells³⁹. All major CTC markers that are known to be critical for the assembly of the CTC-monocyte (FIG. 12C-12D), the CTC-platelet (FIG. 12E-12F; FIG. 13A-13F) and the CTC-NK cell (FIG. 12G-12H) synapses were expressed at significantly higher levels in the autonomous WT cells compared to their GIV-KO counterparts exclusively in 0% FBS conditions. These findings suggest that autonomous WT, but not the autonomy-impaired GIV-KO cells are likely to be able to mount an immune evasion response and by escaping phagocytosis by monocytes, triggering platelet aggregation and activation which shields CTCs from NK cells, and finally, evade cytolytic killing by NK cells.

Besides immune evasion, CTC-neutrophil interactions are known to induce the expression of CTC genes that outline cell cycle progression, leading to more efficient metastasis formation³⁹. This neutrophil-related pro-proliferative signature was highly expressed in the autonomous WT cells but suppressed in the autonomy-impaired GIV-KO cells upon serum depravation (FIG. 12I-12J). A similar pattern was seen also for the universal proliferation marker gene, MKI67 (FIG. 12K), and two other gene sets (Gene Set Enrichment Analysis (GSEA)) for cell cycle progression, KEGG and BIOCARTA (FIG. 13G-H).

These findings suggest that the autonomous state in serum-restricted condition is associated with three key CTC properties that are essential for metastasis, i.e., plasticity, immune evasion, and proliferative potential.

It will be understood from the foregoing description that various modifications and changes may be made in the various embodiments of the present disclosure without departing from their true spirit. The description provided herein is intended for purposes of illustration only and is not intended to be construed in a limiting sense. Thus, while the presently disclosed inventive concepts have been described herein in connection with certain embodiments so that aspects thereof may be more fully understood and appreciated, it is not intended that the presently disclosed inventive concepts be limited to these particular embodiments. On the contrary, it is intended that all alternatives, modifications and equivalents are included within the scope of the presently disclosed inventive concepts as defined herein. Thus the examples described above, which include particular embodiments, will serve to illustrate the practice of the presently disclosed inventive concepts, it being understood that the particulars shown are by way of example and for purposes of illustrative discussion of particular embodiments of the presently disclosed inventive concepts only and are presented in the cause of providing what is believed to be a useful and readily understood description of procedures as well as of the principles and conceptual aspects of the inventive concepts. Changes may be made in the construction and formulation of the various components and compositions described herein, the methods described herein or in the steps or the sequence of steps of the methods described herein without departing from the spirit and scope of the presently disclosed inventive concepts.

### REFERENCES

1. A. W. Lambert, D. R. Pattabiraman, R. A. Weinberg, Emerging Biological Principles of Metastasis. Cell 168, 670-691 (2017).
2. C. L. Chaffer, R. A. Weinberg, A perspective on cancer cell metastasis. Science 331, 1559-1564 (2011).
3. S. A. Joosse, T. M. Gorges, K. Pantel, Biology, detection, and clinical implications of circulating tumor cells. EMBO Mol Med 7, 1-11 (2015).
4. Z. Ju et al., Telomere dysfunction induces environmental alterations limiting hematopoietic stem cell function and engraftment. Nat Med 13, 742-747 (2007).
5. S. Ju et al., Detection of circulating tumor cells: opportunities and challenges. Biomark Res 10, 58 (2022).
6. M. B. Amin et al., The Eighth Edition AJCC Cancer Staging Manual: Continuing to build a bridge from a population-based to a more “personalized” approach to cancer staging. CA Cancer J Clin 67, 93-99 (2017).
7 R. Y. Ebright et al., Deregulation of ribosomal protein expression and translation promotes breast cancer metastasis. Science 367, 1468-1473 (2020).
8. B. M. Szczerba et al., Neutrophils escort circulating tumour cells to enable cell cycle progression. Nature 566, 553-557 (2019).
9. M. Bulfoni et al., In patients with metastatic breast cancer the identification of circulating tumor cells in epithelial-to-mesenchymal transition is associated with a poor prognosis. Breast Cancer Res 18, 30 (2016).
10. M. Yu et al., Circulating breast tumor cells exhibit dynamic changes in epithelial and mesenchymal composition. Science 339, 580-584 (2013).
11. S. Wu et al., Classification of circulating tumor cells by epithelial-mesenchymal transition markers. PLOS One 10, e0123976 (2015).
12. X. Liu et al., Epithelial-type systemic breast carcinoma cells with a restricted mesenchymal transition are a major source of metastasis. Sci Adv 5, eaav4275 (2019).
13. A. M. Dujon et al., Is There. Cancers (Basel) 13, (2021).
14. H. Thangavel et al., A CTC-Cluster-Specific Signature Derived from OMICS Analysis of Patient-Derived Xenograft Tumors Predicts Outcomes in Basal-Like Breast Cancer. J Clin Med 8, (2019).
15. E. Schuster et al., Better together: circulating tumor cell clustering in metastatic cancer. Trends Cancer 7, 1020-1032 (2021).
16. N. Aceto et al., Circulating tumor cell clusters are oligoclonal precursors of breast cancer metastasis. Cell 158, 1110-1122 (2014).
17. R. Taftaf et al., ICAM1 initiates CTC cluster formation and trans-endothelial migration in lung metastasis of breast cancer. Nat Commun 12, 4867 (2021).
18. C. Donato et al., Hypoxia Triggers the Intravasation of Clustered Circulating Tumor Cells. Cell Rep 32, 108105 (2020).
19. Z. Diamantopoulou et al., The metastatic spread of breast cancer accelerates during sleep. Nature 607, 156-162 (2022).
20. I. Baccelli et al., Identification of a population of blood circulating tumor cells from breast cancer patients that initiates metastasis in a xenograft assay. Nat Biotechnol 31, 539-544 (2013).
21. D. Hanahan, R. A. Weinberg, The hallmarks of cancer. Cell 100, 57-70 (2000).
22. T. S. Fedi P, Aaronson S A, Cancer Medicine. B. R. C. Holland J. F, Morton D.L, Frei E, Kufe D. W and Weichselbaum R. R, Ed., Growth factors (Williams and Wilkins, Baltimor, MD, 1997).
23. M. Chigira, K. Noda, H. Watanabe, Autonomy in tumor cell proliferation. Med Hypotheses 32, 249-254 (1990).
24. A. A. A. E. -H. Lingxia Qiao, I-Chung Lo, Saptarshi Sinha, Krishna K. Midde, Tony Ngo, Nicolas Aznar, Inmaculada Lopez-Sanchez, Vijay Gupta, Marilyn G. Farquhar, Padmini Rangamani, Pradipta Ghosh. (2021).
25. I. C. Lo et al., Activation of Gαi at the Golgi by GIV/Girdin imposes finiteness in Arf1 signaling. Dev Cell 33, 189-203 (2015).
26 P. G. Lingxia Qiao, Padmini Rangamani. (bioRxiv, 2022).
27 K. Liu et al., Evaluating cell lines as models for metastatic breast cancer through integrative analysis of genomic data. Nat Commun 10, 2138 (2019).
28. P. Ghosh, Heterotrimeric G proteins as emerging targets for network based therapy in cancer: End of a long futile campaign striking heads of a Hydra. Aging (Albany NY) 7, 469-474 (2015).
29. K. Takahashi, S. Yamanaka, Induction of pluripotent stem cells from mouse embryonic and adult fibroblast cultures by defined factors. Cell 126, 663-676 (2006).
30. T. M. Malta et al., Machine Learning Identifies Stemness Features Associated with Oncogenic Dedifferentiation. Cell 173, 338-354.e315 (2018).
31. J. Yang et al., Guidelines and definitions for research on epithelial-mesenchymal transition. Nat Rev Mol Cell Biol 21, 341-352 (2020).
32. M. S. Brown et al., Phenotypic heterogeneity driven by plasticity of the intermediate EMT state governs disease progression and metastasis in breast cancer. Sci Adv 8, eabj8002 (2022).
33. D. P. Cook, B. C. Vanderhyden, Transcriptional census of epithelial-mesenchymal plasticity in cancer. Sci Adv 8, eabi7640 (2022).
34. M. C. Guadamillas, A. Cerezo, M. A. Del Pozo, Overcoming anoikis—pathways to anchorage-independent growth in cancer. J Cell Sci 124, 3189-3197 (2011).
35. X. Zhu et al., In vivo flow cytometry reveals a circadian rhythm of circulating tumor cells. Light Sci Appl 10, 110 (2021).
36. E. Ozkumur et al., Inertial focusing for tumor antigen-dependent and-independent sorting of rare circulating tumor cells. Sci Transl Med 5, 179ra147 (2013).
37. A. Shinde et al., Spleen Tyrosine Kinase-Mediated Autophagy Is Required for Epithelial-Mesenchymal Plasticity and Metastasis in Breast Cancer. Cancer Res 79, 1831-1843 (2019).
38. S. Khoshbakht, S. Azimzadeh Jamalkandi, A. Masudi-Nejad, Involvement of immune system and Epithelial-Mesenchymal-Transition in increased invasiveness of clustered circulatory tumor cells in breast cancer. BMC Med Genomics 14, 273 (2021).
39. M. P. Ward et al., Platelets, immune cells and the coagulation cascade; friend or foe of the circulating tumour cell? Mol Cancer 20, 59 (2021).
40. P. Aouad et al., Epithelial-mesenchymal plasticity determines estrogen receptor positive breast cancer dormancy and epithelial reconversion drives recurrence. Nat Commun 13, 4975 (2022).
41. P. Balcik-Ercin, L. Cayrefourcq, R. Soundararajan, S. A. Mani, C. Alix-Panabières, Epithelial-to-Mesenchymal Plasticity in Circulating Tumor Cell Lines Sequentially Derived from a Patient with Colorectal Cancer. Cancers (Basel) 13, (2021).
42. K. J. Cheung et al., Polyclonal breast cancer metastases arise from collective dissemination of keratin 14-expressing tumor cell clusters. Proc Natl Acad Sci USA 113, E854-863 (2016).
43. R. D. Z. Mullins, A. Pal, T. F. Barrett, M. E. Heft Neal, S. V. Puram, Epithelial-Mesenchymal Plasticity in Tumor Immune Evasion. Cancer Res 82, 2329-2343 (2022).
44. P. Chakraborty et al., Analysis of immune subtypes across the epithelial-mesenchymal plasticity spectrum. Comput Struct Biotechnol J 19, 3842-3851 (2021).
45. A. Dongre, R. A. Weinberg, New insights into the mechanisms of epithelial-mesenchymal transition and implications for cancer. Nat Rev Mol Cell Biol 20, 69-84 (2019).
46. M. Dhar et al., Functional profiling of circulating tumor cells with an integrated vortex capture and single-cell protease activity assay. Proc Natl Acad Sci USA 115, 9986-9991 (2018).
47. S. Ghannoum et al., DIscBIO: A User-Friendly Pipeline for Biomarker Discovery in Single-Cell Transcriptomics. Int J Mol Sci 22, (2021).
48. P. Wee, Z. Wang, Epidermal Growth Factor Receptor Cell Proliferation Signaling Pathways. Cancers (Basel) 9, (2017).
49. D. C. Voon et al., EMT-induced stemness and tumorigenicity are fueled by the EGFR/Ras pathway. PLOS One 8, e70427 (2013).
50. A. A. Changavi, A. Shashikala, A. S. Ramji, Epidermal Growth Factor Receptor Expression in Triple Negative and Nontriple Negative Breast Carcinomas. J Lab Physicians 7, 79-83 (2015).
51. F. J. Esteva et al., Clinical utility of serum HER2/neu in monitoring and prediction of progression-free survival in metastatic breast cancer patients treated with trastuzumab-based therapies. Breast Cancer Res 7, R436-443 (2005).
52. R. E. Payne et al., Measurements of EGFR expression on circulating tumor cells are reproducible over time in metastatic breast cancer patients. Pharmacogenomics 10, 51-57 (2009).
53. G. Kallergi et al., Phosphorylated EGFR and PI3K/Akt signaling kinases are expressed in circulating tumor cells of breast cancer patients. Breast Cancer Res 10, R80 (2008).
54. X. Liu et al., EGFR inhibition blocks cancer stem cell clustering and lung metastasis of triple negative breast cancer. Theranostics 11, 6632-6643 (2021).
55. L. Bruser, S. Bogdan, Adherens Junctions on the Move-Membrane Trafficking of E-Cadherin. Cold Spring Harb Perspect Biol 9, (2017).
56. R. B. Hazan, L. Norton, The epidermal growth factor receptor modulates the interaction of E-cadherin with the actin cytoskeleton. J Biol Chem 273, 9078-9084 (1998).
57. T. T. Kwan et al., A Digital RNA Signature of Circulating Tumor Cells Predicting Early Therapeutic Response in Localized and Metastatic Breast Cancer. Cancer Discov 8, 1286-1299 (2018).
58. N. S. Amer Ali Abd El-Hafeez, Anirban Chakraborty, Jason Ear, Suchismita Roy, Pranavi Chamarthi, Navin Rajapakse, Soumita Das, Kathryn E. Luker, Tapas K. Hazra, Gary D. Luker, Pradipta Ghosh. (2021).
59. S. P. Cavnar et al., Modeling selective elimination of quiescent cancer cells from bone marrow. Neoplasia 17, 625-633 (2015).
60. M. C. Smith et al., CXCR4 regulates growth of both primary and metastatic breast cancer. Cancer Res 64, 8604-8612 (2004).
61. S. Borowicz et al., The soft agar colony formation assay. J Vis Exp, e51998 (2014).
62. J. M. Buschhaus et al., Effects of iron modulation on mesenchymal stem cell-induced drug resistance in estrogen receptor-positive breast cancer. Oncogene 41, 3705-3718 (2022).
63 J. M. Buschhaus et al., Targeting disseminated estrogen-receptor-positive breast cancer cells in bone marrow. Oncogene 39, 5649-5662 (2020).
64. A. C. Stacer et al., Endothelial CXCR7 regulates breast cancer metastasis. Oncogene 35, 1716-1724 (2016).
65. R. Edgar, M. Domrachev, A. E. Lash, Gene Expression Omnibus: NCBI gene expression and hybridization array data repository. Nucleic Acids Res 30, 207-210 (2002).
66. T. Barrett et al., NCBI GEO: archive for functional genomics data sets—-update. Nucleic Acids Res 41, D991-995 (2013).
67. T. Barrett et al., NCBI GEO: mining tens of millions of expression profiles—database and tools update. Nucleic Acids Res 35, D760-765 (2007).
68. L. A. Peters et al., A functional genomics predictive network model identifies regulators of inflammatory bowel disease. Nat Genet 49, 1437-1449 (2017).
69. P. Dalerba et al., Single-cell dissection of transcriptional heterogeneity in human colon tumors. Nat Biotechnol 29, 1120-1127 (2011).
70. P. Dalerba et al., CDX2 as a Prognostic Biomarker in Stage II and Stage III Colon Cancer. N Engl J Med 374, 211-222 (2016).
71. J. P. Volkmer et al., Three differentiation states risk-stratify bladder cancer into distinct subtypes. Proc Natl Acad Sci USA 109, 2078-2083 (2012).
72. D. Sahoo, D. L. Dill, R. Tibshirani, S. K. Plevritis, Extracting binary signals from microarray time-course data. Nucleic Acids Res 35, 3705-3712 (2007).
73. A. Fabregat et al., The Reactome Pathway Knowledgebase. Nucleic Acids Res 46, D649-D655 (2018).
74. B. Jassal et al., The reactome pathway knowledgebase. Nucleic Acids Res 48, D498-D503 (2020).

1. A method for analyzing growth signaling autonomy, the method comprising:
defining at least one key feature of an autonomous cell line in a biological sample from a subject; and
assessing the role of the at least one key feature of the autonomous cell line during tumor progression.

2. The method of claim 1, wherein the at least one key feature is a transcriptome, a proteome, and/or a phenome.

3. The method of claim 1, wherein growth signaling autonomy induces a presence of circulating tumor cells (CTCs).

4. The method of claim 3, wherein the presence of CTCs indicates an increased likelihood of metastases.

5. The method of claim 4, wherein the tumor is breast cancer.

6. A method for determining the likelihood of tumor metastases, the method comprising:
performing RNA sequencing on a biological sample from a subject to identify differentially expressed genes in autonomous cells;
determining an expression composite score for each the differentially expressed genes; and
using the composite score to assess the autonomy of the cells;
wherein autonomous cells are more likely to metastasize.

7. The method of claim 6, wherein autonomy induces a presence of circulating tumor cells (CTCs).

8. The method of claim 7, wherein the presence of CTCs indicates an increased likelihood of metastases.

9. The method of claim 8, wherein the tumor is breast cancer.

10. A method of treatment of a cancer, comprising determining the likelihood of tumor metastases according to claim 6, and administering to a subject in need thereof determined to have an increased likelihood of tumor metastasis an effective amount of a treatment for the cancer.

11. The method of claim 10, wherein the cancer is breast cancer.
